(19) United States
(12) Patent Application Publication
(10) Pub. No.: US 2004/0055896 A1
(43) Pub. Date: Mar. 25, 2004

(54) Biocidal solution

(75) Inventors: David Anderson, Bryn Mawr, PA (US); Fred Pfleger, Voorhees, NJ (US); Paul Eachus, Pittsgrove Township, NJ (US); Michelle Porcelli, Washington Township, NJ (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: STERILOX TECHNOLOGIES, INC.

(21) Appl. No.: 10/251,343

(22) Filed: Sep. 20, 2002

(51) Int. Cl. C02F 1/461

(52) U.S. Cl. 205/701; 205/742; 205/746; 204/263

(57) ABSTRACT

A biocidal solution is provided having a pH of from 5 to 7 and an available free chlorine content of from 500 to 1,000 ppm. A method is also provided for producing the biocidal solution in an electrolytic cell or cells having anode and cathode chambers which produce respective anolyte and catholyte solutions, wherein the biocidal activity of the biocidal solution is conferred on the solution primarily in the anode chamber. The method includes the steps of supplying the cell or cells with a solution having a salt concentration of 2.0 to 5.0 g/L, such that the solution passes through the anode chamber at a flow rate per anode surface area of 1.25×10³ to 2.75×10³ L hr⁻¹m⁻², and applying current to the cell or cells sufficient to produce a biocidal solution having the above free chlorine content and pH.

### BACKGROUND OF THE INVENTION

The present invention relates to biocidal solutions and to a method of producing a biocidal solution.

The electrolysis of concentrated brine solutions to prepare chlorine gas and caustic soda has been known since the time of Michael Faraday. This process typically uses saturated brine solutions (350 g/L) and current densities of greater than 3-8 kAm⁻².

Nowadays, the electrolysis of brine solutions is used to produce biocidal solutions. Such biocidal solutions have numerous commercial uses from sterilising or disinfecting drinking water, food and food preparation apparatus to sterilising or disinfecting medical apparatus. These solutions are also often called ‘superoxidised water solutions’. The pH, redox potential, salt concentration and available free chlorine (AFC) content of these solutions vary according to the purpose of solution, the equipment in which it is produced and currently held theories as to what types of solution are most effective. The solutions are biocidal against a wide range of bacteria, fungi, viruses and spores. There is an ongoing need for the development of further and more effective biocidal solutions that can be cost effectively produced.

Recent advances in the electrolytic production of biocidal solutions have been based on the commonly held view that a combination of low salt concentration (for example less than 10 g/L) and low current density led to better “activation” of a biocidal solution. Further, although a solution of high AFC content would be expected to be biocidal, such solutions were generally considered inappropriate for use as sterilising solutions due to corrosion and other damage that they would cause to the product being sterilised or disinfected.

“Activation” is used herein as a term to describe the biocidal effectiveness of a solution without necessarily knowing the exact mechanisms by which it is effective. As pH, redox potential, salt concentration, AFC and chemical composition of such solutions can all be varied, and because these factors do not necessarily impact on all bacteria, fungi, viruses and spores in the same manner due to the different biological makeup of such microorganisms, the reason why a specific biocidal solution is particularly effective against a variety of bacteria, fungi, viruses and/or spores will not always be known. The term “activation” relates to this concept.

It is known that the rate of an electrochemical reaction is generally proportional to current within certain limits of current. Therefore the current (and thus the oxidation of chloride ions to chlorine) and flow of chloride ions through an electrolytic cell may be set appropriately to produce an output solution having a predetermined level of AFC. The desired current will depend not only on the type of cell being used, for example, the material from which the electrodes are made and the various rare metals used to provide active coatings on the electrodes, but also the size of the cell, and in particular, the size of the anode. Current density is a measure of the current per unit surface area of the anode in contact with the electrolyte. As mentioned above, although it is possible to produce solutions of particular desired characteristics, due to the varied properties of such solutions and the variety of organisms against which any solution is to be biocidal and the different biological make up of each organism, it is not possible to appreciate the actual effectiveness of any such solution in practice until the solution has been tested.

Authors have described the preparation of biocidal solutions by the electrolysis of low salt concentration solutions, with the formation of AFC and other potential oxidant species. The electrolysis is carried out in either flat plate cells, or concentric cells. Typically, salt concentrations of 1 to 3 g/L are used with current densities below 1 kAm⁻². AFC of the solutions are between 10 and 400 ppm as free Chlorine.

GB 2 352 728 describes the electrolysis of a solution of 3 to 5 g/L sodium chloride with a current of 7 to 9 A (current density 0.8 kAm⁻²) to prepare biocidal solutions of 100 to 400 ppm AFC.

WO 98/42625, EP 838 434 and WO 98/12144 describe the preparation of biocidal solutions by the electrolysis of a reasonably concentrated or saturated (up to 250 g/L) solution of sodium chloride to form chlorine gas, and dissolving the chlorine gas in water.

EP 0 832 850 discloses a process of electrolysing dilute brine solutions with no specific current density. Flow rates are high (250 L/hr) and the only output parameters which control the biocide output specification are pH and redox potential. Work conducted by the Applicant of the present application has shown that redox potential is not a good predictor of biocidal activity.

U.S. Pat. No. 5,731,008 describes the preparation of “electrically hydrolyzed salines” as biocides, with active chlorine species content between 10 and 100 ppm.

EP 0 792 584 describes the preparation of biocidal solutions of pH 3 or less, and hypochlorous acid concentrations of 2 ppm.

U.S. Pat. No. 5,628,888 is also useful as background to the present invention but there is no specific disclosure of current density, output AFC or flow rate.

GB 2 253 860 discloses an electrolysis process with low current density (0.1 kAm⁻²), low charge per unit volume and therefore low biocidal strength output.

Accordingly, at the date of the applicant's invention, the state of the art taught that superoxidised water solutions having biocidal activity had to be prepared by the electrolysis of solutions containing low concentrations of sodium chloride at low current densities, to prepare highly “activated” solutions. There was no obvious reason to alter the above parameters, or to believe that a process using significantly higher current densities would be useful in preparing solution of high AFC content and pH 5 to 7 that would be very biocidal. Further, a reasonably high flow was used by the prior art processes described above, in order to achieve the relatively low degrees of activation required in reasonable production volumes.

With the above documents as a base and with the applicants own experience in the art, the applicant set out to produce an improved biocidal solution generating process or at least to provide the public with a useful choice.

The applicant first tried to produce high AFC concentrations through the use of high input brine concentrations, for example greater than 6 g/L. This proved to be impractical for the end users due to the requirement for high end user input (i.e. the frequent addition of large quantities of salt), the corrosive nature of the solutions and the requirement for large storage containers for salt in clean environments, (due to the large volume of salt required). These high salt feed streams also caused the flow rates of the solutions through cells operating at low current density to be too low for practical purposes. Although the most straight forward manner of producing solutions with high (e.g. 400-600 ppm) AFC content solutions was to use high salt concentration solutions, a further problem that needed to be addressed was the low and therefore impractical shelf life of a solution prepared using high concentration salt solutions.

Low concentration salt solutions used in the prior art processes did produce stable solutions of low corrosion potential but the output solutions were low in AFC. The output solutions therefore did not have acceptable biocidal activity to meet the current regulations across all the world markets.

The Applicant came to believe that the only manner in which the high AFC solutions could be reliably and economically produced was to take the low concentration salt solutions and the relatively high flow rates described in the state of the art and to increase the current density beyond the conventionally accepted limits. Such a concept was not known in the art, nor had it even been considered.

Upon testing of flow rates per anode surface area in the range of 1.25×10³ to 2.75×10³ Lhr⁻¹m⁻², the Applicant found that the reaction that produced the active species (consisting primarily of AFC) was driven further to completion. The term “anode surface area” as used herein means the surface area of the anode, or where multiple anodes are used of all anodes, that are active and in contact with the anolyte, unless the context otherwise requires The Applicant was also surprised to find that the solutions could also be produced in timeframes that were commercially feasible and the solutions had acceptable stability. Once the solution parameters were established, efficacy testing was performed to determine if the increase in conversion and higher concentration of active ingredient did correlate to an increase in activity of the solution. The Applicant was pleased to find that the solutions were particularly effective. An example of the efficacy of the solutions is provided later in the Detailed Description of the Invention

Accordingly, the Applicant has discovered a principle that if a high current density were applied to a solution of higher salt concentration than was typically used in the art for production of biocidal solutions (but still low in salt concentration in terms of the amount of salt that can be loaded into an aqueous solution), and that if a flow rate was used that was lower than that typically used in the art, a particularly effective biocidal solution could result that could still be of salt concentration and pH range low enough for minimal corrosion and safe handling.

It is an object of the present invention to provide an improved biocidal solution and/or method of producing a biocidal solution, or at least to provide the public with a useful choice.

### SUMMARY OF THE INVENTION

In order to carry the principle of the Applicant's invention into effect, and in a first aspect, the present invention provides a biocidal solution having a pH of from 5 to 7 and an available free chlorine content of from 500 to 1000 ppm

In a second aspect, the present invention provides a method of producing a biocidal solution in an electrolytic cell(s), said cell(s) having an anode chamber(s) and a cathode chamber(s) which produce respective anolyte and catholyte solutions, the biocidal activity of the biocidal solution being conferred to the solution primarily in the anode chamber, the method including supplying to the cell(s) a solution with a salt concentration of 2.0 to 5.0 g/L such that the solution passes through the anode chamber at a flow rate per anode surface area of 1.25×10³ to 2.75×10³ L hr⁻¹m⁻² and applying current to the cell(s) sufficient to produce a biocidal solution with an available free chlorine content of 500 to 1000 ppm and a pH of from 5 to 7.

### DETAILED DESCRIPTION OF THE PREFERRED EMBODIMENTS

A description of preferred embodiments of the invention follows. It is to be understood that this is a description of the preferred embodiment and that variations can of course be made with out deviating from the scope of the invention. Such variations will be appreciated by those of skill in the art.

In order that the invention may be more readily understood, reference will now be made, by way of example, to the accompanying figures, in which;

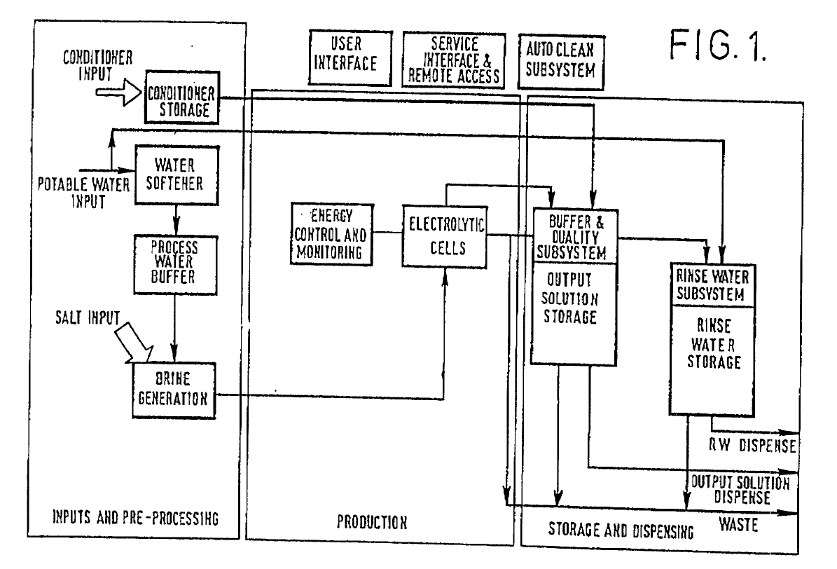

FIG. 1 shows an embodiment of the invention in schematic outline;

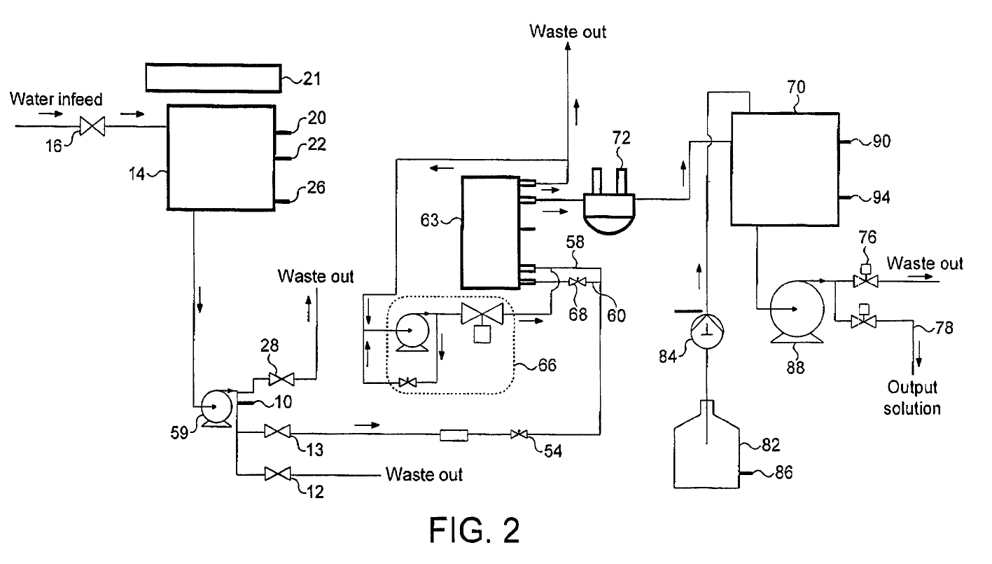

FIG. 2 is a detailed view of the flow diagram of FIG. 1;

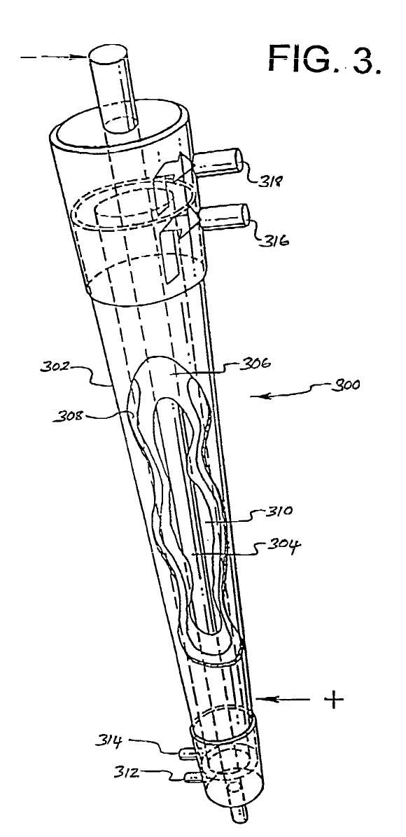

FIG. 3 shows one form of electrolytic cell for use in the present invention and incorporated in the flow diagrams of FIGS. 1 and 2.

Referring first to FIG. 1, the schematic outline of the invention is broken down into three main processing stages, namely an inputs and pre-processing stage, a production stage and a storage and dispensing stage. Whilst referred to as stages, it will of course be appreciated that the process of the invention may be carried out continuously.

In the first stage (inputs and pre-processing), there is an input of potable water which, for the purpose of generating water for use in the electrolytic cell, is first passed through a water softener zone where excessive magnesium and calcium ions are removed. The softened water is then passed as process water for use in the production of brine.

The first stage also includes a salt (typically NaCl) input, usually of vacuum dried crystalline salt which is commercially produced to a consistent standard, to a brine generation zone where a dilute salt solution is made up from the salt and the softened water.

A further input (conditioning input) is provided for additional agents, such as a corrosion inhibitor, used to condition output solution produced by the process. The conditioner is passed to a conditioner storage zone where it is held until required.

A saline solution of substantially constant concentration is produced by addition of a known quantity of salt to a known quantity of softened water to achieve the desired concentration. The resulting constant salinity saline solution is passed to one or more electrolytic cells like that of FIG. 3 for example, each including cathode and anode chambers, and across which a substantially constant electric current is applied. The applied electric current is maintained constant via an energy control and monitoring zone.

Catholyte and anolyte are produced from the cathode and anode chambers respectively as a result of the electrochemical treatment of the saline solution in the cells. Anolyte and a portion of catholyte which is not recirculated to the anode chamber are both dealt with in the third (storage and dispensing) stage. In particular, catholyte which is not recirculated is directed to waste and anolyte, otherwise referred to as output solution, is passed to a buffer and quality subsystem. The output solution is tested in the buffer and quality subsystem and, if it fails to meet the quality standards, it is also directed to waste. If the output solution falls within specification, a quantity of conditioner, such as a corrosion inhibitor, is added to it in the buffer subsystem and the output solution is then permitted to pass either into an output solution storage zone from where it is subsequently dispensed for use.

Provision is also made for discharging output solution from the output solution storage zone to waste.

Information on the various processing stages and the ability to interact with the process is provided by means of a user interface and a service interface.

There may also be provided an autoclean subsystem to permit cleaning of the system, either at regular intervals or whenever convenient.

Furthermore a rinse water subsystem may also be provided, such as described in GB 2 352 728 (Sterilox Medical (Europe) Limited), the subject matter of which is herein incorporated by reference.

FIG. 2 is a flow diagram or “hydraulic map” showing the preferred embodiment in more detail. Potable water is passed through an external water softener containing a cation exchange resin (not shown) thereby exchanging hardness ions of calcium and magnesium onto the resin and releasing sodium ions into the water and thence into a process water tank 14 which includes a plurality of level detectors for monitoring and controlling the process water level in it. Level detector 20 is a safety device which is activated only when the process water in the tank reaches a predetermined extra high level to stop the charging of the tank with process water and raise an alarm. Level detector 22 ensures that the tank has the correct volume of softened water to prepare the appropriate concentration saline solution. Water will begin to re-charge the tank 14 when the process water drops below the low level, determined by level detector 26 and at the end of the production of one batch of biocidal solution. The tank 14 also includes a valve 28 which allows liquid to be drained. Furthermore, the tank 14 is designed to comply with local regulations, such as the class A air break requirements as required in the United Kingdom by Building Regulations Bylaw 11

Dilute salt solution is made-up and stored in the tank 14. To make up the dilute salt solution, vacuum dried crystalline salt (BS998:1990) is added to the tank 14 via a dispensing wheel 21. The dispensing wheel contains many tablets of known mass of salt, a pre-determined number of which are dispensed through a hole in the top of tank 14 at the start of each biocidal solution production cycle.

Softened water is fed through the valve 16 and automatically fills the tank 14 until the high level switch 22 is activated. Salt in the tank 14 dissolves in the water to produce a dilute salt solution.

The flow rate of the saline solution as it is pumped by pump 59 towards the electrolytic cell pack 63 is monitored by a flow meter (not shown) and if necessary is modulated by a flow regulator in the form of an orifice plate 54. The flow rate is changed simply by changing the size of the orifice in the plate. Different orifice plates may be chosen to suit different water types.

Saline solution is monitored by a sensor 10. The sensor 10 ascertains whether the incoming water is at a temperature within the range under which the process can reasonably operate, namely between about 5 and 35° C. Other parameters such as the incoming water's pressure, softness, alkalinity, pH, conductivity and microbial count can also be monitored by the sensor 10 to establish that it falls within acceptable levels for the process. A person of skill in the art will be able to appreciate when the incoming water is not suitable for processing according to the invention.

If the sensor 10 detects that the properties of the incoming saline water do not fall within acceptable limits required by the specification, the water is diverted through a waste discharge manifold (not shown) to a drain via valve 12. On the other hand, if the incoming softened process water is in-specification, it is allowed to flow into the cells through valve 13.

The saline solution is then split into two streams 58, 60 before being fed through the cell pack.

In the preferred embodiment the cell pack consists of eight electrolytic cells, with two sets of four cells connected hydraulically in parallel. For simplicity, only one cell is illustrated. In general, the number of cells in the cell pack is determined by the output volume required from the particular system. Each cell has an anode chamber and a cathode chamber(s) and the flow of saline solution is split such that the greater portion is fed to the anode chamber(s) and the lesser portion is fed to the cathode chamber(s). In the preferred embodiment, approximately 90% of the saline solution is passed through the anode chamber(s) with the remainder being passed through the cathode chamber(s). The flow rate of saline solution through the cathode chamber is much lower than for the anode chamber and the pressure in the cathode chamber is also lower. The flow rate of the saline solution into the cathode chamber, which also has an influence on the pH of the output solution, is controlled by a flow regulator 68. The flow regulator 68 can be manually adjusted if there is a variation in input water quality.

In general, preferably, the flow rate supplied to the anode(s) is from 50% to 95%, inclusive of all intermediate values, of the solution applied to the electrolytic cell pack. Supplying such a volume to the anode allows large volumes of in-specification output solution to be produced. More preferably, the flow rate to the anode(s) is from 85 to 95% of the solution supplied to the electrolytic cell.

As the saline solution flows through the electrolytic cells, a fixed current of from 15 to 25 amps, preferably 18-19 amps, is applied to each cell causing electrolysis of the saline solution thereby generating available free chlorine in the resulting anolyte, elsewhere generally referred to as the output solution. In order to produce output solution at a relatively neutral pH, namely between 5 and 7, the pH of the output solution may be at least partially controlled by dosing a portion of the catholyte to the inlet stream 58 for the anode chambers. The catholyte is dosed to the inlet stream 58 by an adjustable pump and valve system 66 and the dosing rate is increased or decreased to achieve the target pH. The remaining catholyte which is not dosed into the input stream 58 for the anode chambers is directed to waste, if necessary diluting it prior to disposal. As just described, in the preferred embodiment the catholyte is dosed into the anode stream 58 before this stream enters the anode. However, the catholyte can also be dosed into the anode stream after it has been electrolysed.

The pH of the biocidal solutions of the preferred embodiments of the invention are preferably in the range of 5 to 7, inclusive of all intermediate values. Values above this result in low biocidal efficacy. Values below this result in corrosive solutions, which are toxicologically less benign and present a potential hazard due to the evolution of chlorine gas. In order to provide a particularly effective solution it is desirable to have a pH range of 5.5 to 6.8. More preferable is a pH range of 6.0 to 6.5.

The pH of the output solution in the flow systems going to tank 70 are measured by a pH meter 72. If the pH does not fall within the desired parameters, a valve 76 is opened and the contents of the tank 70 are drained to waste. The contents of the tank 70 are drained to waste in any event if they have remained in the tank for longer than the storage lifetime of the particular solution. The pH meter 72 is linked to the pump and valve system 66 to adjust the level of catholyte dosed to the anode chambers thereby enabling the pH of the output solution to be adjusted to bring the output solution within the desired pH range. If the pH of the output solution is determined to fall within the desired parameters, confirming that the output solution has the necessary biocidal efficacy, the valve 76 is kept closed and the output solution is allowed to fill the tank 70 until it reaches the high level. The person skilled in the art will appreciate that other properties of the output solution, such as redox potential or AFC, could form the basis of the measurement and control system consisting of probe 72 and adjustable pump and valve system 66.

Provided the pH meter 72 confirms that the output solution (78) has the desired parameters, a corrosion inhibitor, such as a mixture of sodium hexametaphosphate and sodium molybdate, is dosed as a solution from a storage container 82 into the output solution in the tank 70 by a pump 84. A sensor 86 is mounted in the storage container to monitor low levels of inhibitor and trigger an alert mechanism which alerts the system that there is a need for inhibitor to be supplied to the storage container 82.

In the preferred embodiment, the additive is a corrosion inhibitor and it is added after electrolysis of the solution. An alternative or in some cases additional additive is a surfactant. Additives may be added prior to electrolysis if desired, although the electrolytic activity of the additive, if any, will need to be taken into account in such a situation.

In-specification output solution remains in the tank 70 until a demand for it is received. For example, when it is required for a cycle of a washer-disinfector machine, the system receives a demand signal from a washing machine interface control module triggering operation of a dispensing pump 88. In the preferred embodiment the dispensing pump 88 is rated so that it can supply output solution to washing machine vessels of 25 litre capacity in 180 seconds (1500 litres per hour, 3 bar line pressure). The capacity of the storage tank 70 is therefore such that it too can fulfil the volume requirement.

The storage tank 70 includes various level detectors for monitoring liquid levels in the tank. A level detector 90 is activated by an extra high level of output solution within the tank, raising an alarm and stopping production. Low level detector 94 is activated when the level of the output solution falls to a low level, raising an alarm and preventing further dispensing to the washing machine or other appropriate receptacle. As the output solution is dispensed and after a period of time below the level of detector 94, production of output solution is recommenced.

Once the output solution has been stored for a period determined by the lifetime of acceptable biocidal efficacy hours, it is similarly routed to waste. In this way, output solution which has exceeded its demonstrated shelf life is never dispensed.

Output solution held in the tank 70 may be used to produce bacteria-free rinse water as described in GB 2 352 728. Fresh output solution is dosed at a predetermined rate from the tank 70 to a rinse water storage tank (not shown in FIG. 2) via a pump. Potable water flows into the tank where it is mixed with and dilutes the output solution 78 to a concentration of 2-5%. If the local water supply is of poor quality, a higher concentration of output solution in the rinse water, for example a 5% solution, is preferred. Accordingly, the dosing rate of the pump is determined by the incoming potable water supply and is monitored by a flowmeter.

The volume of solution dispensed to the washer-disinfector may be monitored by a flowmeter, which also is used in ‘no flow’ and leak detection routines linked to the user/service interface (FIG. 1). By automatic monitoring of liquid levels in the tank 70 and by discharging the output solution 78 and rinse water periodically, the system is able to self-adjust to allow it to meet demand at all times. Gases generated by the electrolytic reaction in the cell pack, mainly chlorine, are vented through a carbon filter located above tank 70 to reduce the quantity of chlorine which escapes.

FIG. 3 shows an electrolytic cell 300 as used in the preferred embodiment of the present invention. The cell 300 comprises co-axial cylindrical and rod electrodes 302, 304 respectively, separated by a semi-permeable ceramic membrane 306 co-axially mounted between the electrodes thus splitting the space between the electrodes to form two chambers 308, 310. The cylindrical electrode 302 forming the anode is typically made from commercially pure titanium coated with a ruthenium oxide and iridium oxide-based electrocatalytic (active) coating suitable for the evolution of chlorine from a chloride solution. The anode has a surface area of 0.01 m². The rod electrode 304 forming the cathode is made from titanium and machined from an 8 mm stock bar to a uniform cross-section over its effective length, which is typically about 210 mm±0.5 mm. The semi-permeable ceramic membrane 306 forming a separator and creating the anode and cathode chambers 308 and 310 is composed of aluminium oxide (80%), zirconium oxide (18.5%) and yttrium oxide (1.5%), and has a porosity of about 50-70%, a pore size of 0.3 to 0.5 microns and a wall thickness of 0.5 mm+0.3 mm/−0.1 mm. The ceramic of the membrane 306 is more fully disclosed in the specification of patent application GB 2354478 (Sterilox Medical (Europe) Limited), the subject matter of which is incorporated herein by reference. The cell separator can be made of ceramics other than the aluminium oxide, zirconium oxide and yttrium oxide ceramic described and of any other suitable semi-permeable or ion-selective material.

Generally, the surface area of anode will be largely defined by the quantities of output solution desired to be produced and AFC content desired in that solution. However, in order to provide a system that is of a size appropriate for commercial installation and to produce the quantities of biocidal solution of the invention often required, an anode surface area of 0.065 to 0.095 m² is desirable, inclusive of all intermediate values. Such a surface area can be made up by a number of electrolytic cells working in parallel. An anode area of 0.070 to 0.090 m² is a more useful size, and an anode surface area of 0.075 to 0.085 is even more useful.

The cell 300 is provided with entry passages 312, 314 to permit the saline solution to enter the cell 300 and flow upwards through the anode and cathode chambers 308 and 310 and is discharged as anolyte and catholyte through exit passages 316, 318 respectively. The anolyte containing available free chlorine constitutes the output solution.

As previously described, in the preferred embodiment in order to provide a useful amount of output solution within a reasonable period of time, a group of cells are connected together to form a cell pack 63. For example, a cell pack comprising eight cells connected together in parallel hydraulically and in series electrically is capable of generating about 200 litres/hour of output solution.

In the preferred embodiment of the invention the flow rate is 100 to 220 L/hr. Such a flow rate is applicable to the apparatus described herein. A flow rate of 150 to 210 L/hr is more preferred and a flow rate of 190 to 200 L/hr is even more preferred as these ranges provide the best balance between production of in-specification biocidal solution and current use. The flow rate can also be any value within the expressed ranges.

The person skilled in the art will appreciate that the flow rate can be altered beyond such a range but still produce the solution of the invention by varying the number of cells/surface area of anode. Accordingly, the present invention is better expressed generally in terms of flow rate across the active surface area of the anode; that is, a flow rate per anode surface area of 1.25×10³ to 2.75×10³ Lhr⁻¹m⁻² is desirable to produce the biocidal solution of the invention. The flow rate can also take any value with the aforementioned range. The invention is however even more effective when the flow rate is 1.87×10³ to 2.63×10³ Lhr⁻¹m⁻² and still more effective when the flow rate is 2.31×10³ to 2.56×10³ L hr⁻¹m⁻². Accordingly, these latter flow rates are more preferable. The skilled person will be able to obtain the required current to produce a solution of the invention by setting the flow rate to that just described and varying the current until the solution produced has the specifications of the solution of the invention

A flow rate higher than the flow rates of the invention would require a corresponding increase in current density to maintain the production of a solution of 500-1000 ppm AFC, and such an increase in current density would result in further and unacceptable heating of electrolyte. Further heating of the electrolyte is undesirable because of the associated wasted energy and the associated reduction in compatibility of the output biocidal solution with other components of the biocide generating and dispensing machine, for example the pumps, pipes and valves, or devices which are to be disinfected using the biocidal solution, such as flexible endoscopes. Increased current density beyond this level also unacceptably reduces the durability of the electrodes. At lower flow rates it is not possible to achieve the desired AFC range in a commercially desirable manner since the output of biocidal solution is too low to be useful and requires more anode surface area, increasing the cost of the generating device.

The AFC content of the biocidal solution of the invention is 500 to 1000 ppm, or any value within that range. Preferably the biocidal solution has a AFC content of 550 to 900 ppm. Such a range balances the benefits of a high AFC solution with the flow rates and current required to obtain such a solution. maximising the benefits of high flow with minimising the disadvantages of too much current. An AFC content of 600 to 800 ppm, or even 650 to 750 ppm is even more preferred for the same reason.

The preferred current range for use in the invention is 15 to 25 A, inclusive of all intermediate values. As expressed above, currents lower than this can result in AFC values too low to be useful, and currents higher than this result in unacceptable heating of the solution, energy loss and negative effects on the apparatus used to produce the solution, as mentioned above. A current range of 17 to 22 A is more preferred as this provides a better balance between energy usage and AFC produced, and a current range of 18.5 to 19.5 A is even more preferred for the same reason.

It is desirable that the residual salt concentration of the invention is 2.0 to 4.0 g/L. As the residual salt concentration is related to the input solution salt concentration, the preferred residual salt concentration is high enough to allow an input solution salt concentration that will provide the AFC levels required for the inventive solution, but not much higher so that the corrosive nature of residual salt in the biocidal solution can be minimised. In order to balance these conflicting demands a residual concentration of 3.0 to 3.7 g/L is more preferred.

So as to ensure a commercially desirable product results, preferably 80% of the active species present in the solution at the time of production, are still present 4 hours after production. More preferably 90% will be present 4 hours after production.

A preferred embodiment has an input solution salt concentration range of 2.0 to 5.0 g/L. Such a range was found to effectively produce the specifications required for the biocidal solution of the invention. However, at concentrations lower than 2.0 g/L sufficient quantities of AFC become difficult to produce without encountering other negative factors associated with high current and low flow (such factors have been outlined above). An input solution with a salt concentration above 5 g/L will have the disadvantages mentioned above associated with high salt concentration and low current. Accordingly, it is preferred that the salt concentration of the input solution for use in the invention is 2-5 to 4.5 g/L, more preferably 3.0 to 4.0 g/L, and most preferably 3,4 to 4.6 g/L.

### EXAMPLE

Using the apparatus described in detail above, a dilute saline solution was produced in a tank by adding a fixed known volume of softened water at ambient temperature to 4 tablets of pure vacuum dried salt to generate a saline solution of 3.5 g/L. A biocidal solution was then generated by passing the saline solution through eight electrolytic cells at a fixed flow rate of approximately 200 L/hr. A constant current of 19 A was applied through each cell. The output biocidal solution was stored in a tank until needed.

The table below shows the results of sporicidal testing of biocidal solutions according to AOAC method 966.04. To briefly describe this test, 60 carriers such as porcelain penicyclinders are immersed in a spore suspension for a set time and removed and dried under vacuum. Then, the cylinders are immersed in groups of 5 in the test disinfectant solution for the appropriate exposure time. After exposure the carriers are removed, neutralised and transferred to a growth medium. If no growth is observed at the end of a 21-day incubation period, that cyclinder is considered to be “sterile”. In order to pass the test all 60 cylinders must be “sterile”.

Tests on solutions prepared using processes described in the prior art GB 2 352 728 (C1 and C2 in the table below) show such solutions not to be of sufficient biocidal efficacy. Tests on solutions having an AFC of 600 ppm or greater as developed by the Applicant's new techniques as embodied in the present invention were able to sterilise all sixty cylinders (Example 4 in the table below).

| | Test AFC | Contact | Number of Cylinders |
|---|---|---|---|
| Example | (ppm) | Time | sterilised |
| C1 | 340 | 24 Hours | 36/40 |
| C2 | 400 | 24 Hours | 57/60 |
| 4 | 600 | 24 Hours | 59/60, 60/60, 60/60, 60/60, |
| | | | 60/60, 59/60 |

Although the invention has been particularly described, it should be appreciated that the invention is not limited to the particular embodiments described and illustrated but includes all modifications and variations falling within the scope of the invention as defined in the appended claims.

We claim:

1. A biocidal solution having a pH of from 5 to 7 and an available free chlorine content of from 500 to 1000 ppm.

2. A method of producing a biocidal solution in an electrolytic cell(s), said cell(s) having an anode chamber(s) and a cathode chamber(s) which produce respective anolyte and catholyte solutions, the biocidal activity of the biocidal solution being conferred to the solution primarily in the anode chamber, the method including supplying to the cell(s) a solution with a salt concentration of 2.0 to 5.0 g/L such that the solution passes through the anode chamber at a flow rate per anode surface area of 1.25×10³ to 2.75×10³ Lhr⁻¹m⁻² and applying current to the cell(s) sufficient to produce a biocidal solution with an available free chlorine content of 500 to 1000 ppm and a pH of from 5 to 73.

3. A method as claimed in claim 2 wherein the flow rate is 1.87×10³ to 2.63×10³ Lhr⁻¹m⁻².

4. A method as claimed in claim 2 wherein the flow rate is 2.31×10³ to 2.56×10³ Lhr⁻¹m⁻².

5. A method as claimed in claim 2 wherein the salt concentration is 2.5 to 4.5 g/L

6. A method as claimed in claim 2 wherein the salt concentration is 3.0 to 4.0 g/L

7. A method as claimed in claim 2 wherein the salt concentration is 3.4 to 3.6 g/L.

8. A method as claimed in claim 2 wherein at least a portion of the catholyte is recycled into the anolyte.

9. A method as claimed in claim 2 wherein the current density applied is 1.5 to 2.5 kAM⁻².

10. A method as claimed in claim 2 wherein the current density applied is 1.7 to 2.2 kAm⁻².

11. A method as claimed in claim 2 wherein the current density applied is 1.85 to 1.95 kAm⁻².

12. A method as claimed in claim 2 wherein the available free chlorine content of the solution produced is 550 to 900 ppm.

13. A method as claimed in claim 2 wherein the available free chlorine content of the solution produced is 600 to 800 ppm.

14. A method as claimed in claim 2 wherein the available free chlorine content of the solution produced is 650 to 750 ppm.

15. A method as claimed in claim 2 wherein the salt is NaCl.

16. A method as claimed in claim 2 wherein the cell is a tubular cell.

17. A method as claimed in claim 2 wherein the cell has concentrically arranged anode and cathode chambers.

18. A method as claimed in claim 2 wherein the anode surface area is 0.065 to 0.095 m².

19. A method as claimed in claim 2 wherein the anode surface area is 0.070 to 0.090 m².

20. A method as claimed in claim 2 wherein the anode surface area is 0.075 to 0.085 m².

21. A method as claimed in claim 2 wherein the pH of the biocidal solution is 5.5 to 6.8.

22. A method as claimed in claim 2 wherein the pH of the biocidal solution is 6.0 to 6.5.

23. A method as claimed in claim 2 wherein the solution has a residual salt concentration of 2.0 to 4.0 g/L.

24. A method as claimed in claim 2 wherein the solution has a residual salt concentration of 3.0 to 3.7 g/L

25. A method of claim 2 wherein one or more additives are added to the electrolyte during production of the biocidal solution.

26. A method as claimed in claim 25 wherein the additive is a corrosion inhibitor.

27. A method as claimed in claim 25 wherein the additive is a surfactant.

28. A method as claimed in claim 2 wherein the water of the salt solution has been softened before the salt has been added to it.

29. A method as claimed in claim 2 wherein at least 80% of the active species present in the solution at the time of production are still present 4 hours after production of solution.

30. A method as claimed in claim 2 wherein at least 90% of the active species of the solution are still active 4 hours after production of solution.

31. A method as claimed in claim 2 wherein the flow rate is 100 to 220 L/hr.

32. A method as claimed in claim 2 wherein the flow rate is 150 to 210 L/hr.

33. A method as claimed in claim 2 wherein the flow rate is 185 to 205 L/hr.

34. A method as claimed in claim 2 wherein the biocidal solution is suitable for use in disinfecting food preparation apparatus or food itself.

35. A method as claimed in claim 2 wherein the biocidal solution is suitable for use in disinfecting medical apparatus.

36. A method as claimed in claim 2 wherein from 50% to 95% of the flow rate of the solution supplied to the electrolytic cell is supplied to the anode.

37. A method as claimed in claim 2 wherein between 85% and 95% of the flow rate of the solution supplied to the electrolytic cell is supplied to the anode.

38. A biocidal solution whenever produced by the method of claim 2.

39. Apparatus adapted for producing a biocidal solution according to the method of claim 2.
